(19) United States
(12) Patent Application Publication
(10) Pub. No.: US 2012/0036649 A1
(43) Pub. Date: Feb. 16, 2012

(54) ENZYMATIC TEXTILE BLEACHING COMPOSITIONS AND METHODS OF USE THEREOF

(75) Inventors: Anna-Liisa Auterinen, Espoo (FI); Biancamari Prozzo, Basel (CH); Erwin Redling, Maulberg (DE); Lode Vermeersch, Mullheim (DE); Mee-Young Yoon, Harborside, ME (US)

(73) Assignee: DANISCO US INC., Palo Alto, CA (US)

(21) Appl. No.: 13/063,140

(22) PCT Filed: Sep. 10, 2009
(86) PCT No.: PCT/US09/56499

§ 371 Date: Sep. 15, 2011

Related U.S. Application Data
(60) Provisional application No. 61/095,807, filed on Sep. 10, 2008.
(60) Provisional application No. 61/099,020, filed on Sep. 22, 2008.
(60) Provisional application No. 61/156,593, filed on Mar. 2, 2009.

(51) Int. Cl. D06L 3/11; D06L 3/14; C12N 9/18

(52) U.S. Cl. 8/111; 435/197

(57) ABSTRACT

Described are compositions and methods for enzymatic bleaching of textiles. A perhydrolase enzyme is used in combination with an ester substrate and hydrogen peroxide to produce a peracid for textile bleaching. Textiles bleached by the methods herein exhibit increased dye uptake, decreased textile damage, and/or bulkier softer handle than textiles bleached by conventional chemical bleaching processes.

### PRIORITY

The present application claims priority to U.S. Provisional Patent Application Ser. Nos. 61/095,807, filed on Sep. 10, 2008, 61/099,020, filed on Sep. 22, 2008, and 61/156,593, filed on Mar. 2, 2009, each of which is incorporated by reference in its entirety.

### TECHNICAL FIELD

The compositions and methods relate to the enzymatic bleaching of textiles.

### BACKGROUND

In the processing of textile fibers, yarns and fabrics, a pretreatment or preparation step is typically required to properly prepare the natural materials for further use, in particular, for the dyeing, printing, and/or finishing stages typically required for commercial goods. These textile treatment steps remove impurities and color bodies which exist either naturally or are added to the fibers and/or fabrics during spinning or weaving.

Textile manufacturing typically includes a number of treatments and stages, the most common being de-sizing (i.e., the removal of sizing agents, such as starches, via enzymatic, alkali or oxidative soaking); scouring (i.e., the removal of greases, oils, waxes, pectic substances, motes, protein and fats by contact with a solution of sodium hydroxide at temperatures near boiling); and bleaching (i.e., the removal and lightening of color bodies from textiles by convensional using oxidizing agents, such as hydrogen peroxide, hypochlorite, and chlorine dioxide, or by using reducing agents, such as, sulfur dioxide or hydrosulfite salts). Currently employed bleaching technology involves the use of alkaline hydrogen peroxide bleaching at temperatures in excess of 95° C. Such high temperatures and strong bleaching systems require high energy input and typically produce high pH effluent, which is undesirable from the perspective of environmental sustainability.

There is a need for an effective enzymatic textile bleaching process that minimizes the environmental footprint and costs of textile mills and provides improved fabric strength retention and reduced fiber damage compared to conventional textile bleaching processes. Such an enzymatic bleaching process would preferably operate at a lower pH and lower temperature, decrease the use of caustic chemicals, and be more environmentally friendly than conventional methods.

### BRIEF SUMMARY

The present compositions and methods relate to enzymatic textile bleaching. Use of the textile bleaching compositions and methods produce bleached textiles with decreased textile damage, bulkier softer handle, and/or increased dye uptake when compared to a chemical textile bleaching method.

In one aspect, an enzymatic textile bleaching composition is provided, comprising: (i) a perhydrolase enzyme; (ii) an ester substrate for said perhydrolase enzyme; (iii) a hydrogen peroxide source; (iv) a surfactant and/or an emulsifier; (v) a peroxide stabilizer; (vi) a sequestering agent; and (vii) a buffer that maintains a pH of about 6 to about 8.

In some embodiments, the perhydrolase enzyme comprises the amino acid sequence set forth in SEQ ID NO: 1 or a variant or homolog thereof. In particular embodiments, the perhydrolase enzyme is the S54V variant of SEQ ID NO: 1 (i.e., a variant of SEQ ID NO: 1 having the substitution S54V). In some embodiments, the perhydrolase enzyme comprises (i.e., exhibits) a perhydrolysis to hydrolysis ratio greater than 1. In some embodiments, the perhydrolase enzyme is present at a concentration of about 1 to about 2.5 ppm, for example, about 1.7 ppm.

In some embodiments, the ester substrate is selected from propylene glycol diacetate, ethylene glycol diacetate, triacetin, ethyl acetate, and tributyrin. In a particular embodiment, the ester substrate is propylene glycol diacetate. In some embodiments, propylene glycol diacetate is present in the composition in an amount of about 2,000 to about 4,000 ppm, for example, about 3,000 ppm.

In some embodiments, the hydrogen peroxide source is hydrogen peroxide. In some embodiments, hydrogen peroxide is present at a concentration of about 1,000 to about 3,000 ppm, for example, about 2,100 ppm.

In some embodiments, the surfactant and/or emulsifier comprises a non-ionic surfactant. In one embodiment, the non-ionic surfactant is an alcohol ethoxylate. In one embodiment, the surfactant and/or emulsifier comprises an isotridecanol ethoxylate. In one embodiment, the surfactant and/or emulsifier comprises an alcohol ethoxylate and an isotridecanol ethoxylate. In one embodiment, the composition comprises a surfactant and an emulsifier.

In some embodiments, the enzymatic textile bleaching composition comprises a peroxide stabilizer and/or a sequestering agent. In one embodiment, the peroxide stabilizer is phosphonic acid. In one embodiment, the sequestering agent is polyacrylic acid.

In some embodiments, the composition further comprises a bioscouring enzyme. In some embodiments, the bioscouring enzyme is selected from pectinases, cutinases, cellulases, hemicellulases, proteases, and lipases. In one embodiment, the bioscouring enzyme is a pectinase.

In another aspect, a method for bleaching a textile is provided, comprising contacting the textile with an enzymatic textile bleaching composition as described herein for a length of time and under conditions suitable to permit measurable whitening of the textile, thereby producing a bleached textile, wherein the bleached textile comprises at least one of decreased textile damage, bulkier softer handle, and increased dye uptake when compared to a chemical textile bleaching method that comprises contacting the textile with a chemical textile bleaching composition that does not comprise a perhydrolase enzyme. In some embodiments, the method further comprises hydrolyzing hydrogen peroxide with a catalase enzyme after the bleached textile is produced. In one embodiment, the liquor ratio is about 10:1. In some embodiments, the method is performed in a batch or exhaust process.

In some embodiments, the method provides any of at least about 10, 20, 30, 40, or 50% less weight loss than a chemical bleaching composition that does not comprise a perhydrolase enzyme.

In some embodiments, the method provides a textile capable of increased dye uptake to produce a dyed textile with at least about any of at least about 5, 10, 15, 20, 25, or 30% increased dye depth when compared to a textile treated with a chemical bleaching composition that does not comprise a perhydrolase enzyme.

In some embodiments, the method provides a textile that demonstrated (i.e., exhibits or possesses) reduced pilling propensity when compared to a textile treated with a chemical bleaching composition that does not comprise a perhydrolase enzyme.

In some embodiments, the textile is contacted with the enzymatic textile bleaching composition at a bleaching temperature of about 60° to about 70° C. for a processing time of about 40 to about 60 minutes. In some embodiments, the temperature of the enzymatic textile bleaching composition is raised by about 3° C. per minute from a starting temperature of about 20° to about 40° C. until the bleaching temperature is reached. In one embodiment, the bleaching temperature is about 65° C. and the processing time is about 50 minutes.

In some embodiments, the bleached textile is rinsed with an aqueous composition at a rinsing temperature of about 40° C. to about 60° C. to remove said enzymatic textile bleaching composition. In one embodiment, the rinsing temperature is about 50° C. In one embodiment, rinsing comprises rinsing said bleached textile twice for about 10 minutes for each rinse. In some embodiments, the aqueous composition comprises a catalase enzyme to hydrolyze the hydrogen peroxide.

In another aspect, use of an enzymatic textile bleaching composition for bleaching a cellulose-containing textile is provided, the composition comprising an enzymatic textile bleaching composition as described herein, characterized in that treating the textile with the composition provides improved dye uptake, bulkier softer handle, and/or decreased textile damage as compared to treatment with chemical bleaching.

### DETAILED DESCRIPTION

The present compositions and methods relate to the enzymatic bleaching of textiles using a perhydrolase enzyme. The described enzymatic processes result in textiles with a bulkier softer handle, increased dye uptake, and/or decreased textile damage when compared with a chemical bleaching process. The processes are generally performed at a lower temperature and with a lower rinsing requirement than traditional chemical bleaching processes, resulting in energy and water savings. The effluent from the enzymatic bleaching process also has a lower pH (i.e., <8) than that of a conventional chemical bleaching process (i.e., about 13), thereby reducing the environmental impact of textile bleaching.

Unless otherwise indicated, the practice of the present compositions and methods will employ conventional techniques in the fields of molecular biology (including recombinant techniques), microbiology, cell biology, and biochemistry, which are known to those skilled in the art. Such techniques are describe in the literature, for example, Molecular Cloning: A Laboratory Manual, second edition (Sambrook et al., 1989); Oligonucleotide Synthesis (M. J. Gait, ed., 1984; Current Protocols in Molecular Biology (F. M. Ausubel et al., eds., 1994); PCR: The Polymerase Chain Reaction (Mullis et al., eds., 1994); and Gene Transfer and Expression: A Laboratory Manual (Kriegler, 1990).

Unless otherwise defined, all technical and scientific terms used herein have the same meaning as commonly understood by one of ordinary skill in the art. Singleton, et al., Dictionary of Microbiology and Molecular Biology, second ed., John Wiley and Sons, New York (1994), and Hale & Markham, The Harper Collins Dictionary of Biology, Harper Perennial, NY (1991) provide a general dictionary for reference.

Unless otherwise specified, numeric ranges are inclusive of the numbers defining the range, nucleic acids are written left to right in 5′ to 3′ orientation, and amino acid sequences are written left to right in amino to carboxy orientation. Unless context clearly dictates otherwise, the articles “a,” “an,” and “the” include both singular and plural referents. Unless otherwise specified, any methods and materials similar or equivalent to those described can be used in the practice or testing of the present compositions and methods. All reference cited herein are hereby incorporated by reference.

### Definitions

The following terms and phrases are defined for clarity:

As used herein, the term “bleaching” refers the process of treating a textile material such as a fiber, yarn, fabric, garment or non-woven material to produce a lighter color. Bleaching encompasses the whitening of a textile by removal, modification, or masking of color-causing compounds in cellulosic or other textile materials. Thus, “bleaching” refers to the treatment of a textile for a sufficient length of time and under appropriate pH and temperature conditions to effect a brightening (i.e., whitening) of the textile. Bleaching may be performed using chemical bleaching agent(s) and/or enzymatically generated bleaching agent(s). Examples of suitable bleaching agents include but are not limited to ClO₂, H₂O₂, peracids, NO₂, and the like.

As used herein, the term “bleaching agent” encompasses any moiety/chemical that is capable of bleaching a textile. A bleaching agent may require the presence of a bleach activator. Examples of suitable chemical bleaching agents are sodium peroxide, sodium perborate, potassium permanganate, and peracids. H₂O₂ may be considered a chemical bleaching agent when it has been generated enzymatically in situ. A “chemical bleaching composition” contains one or more chemical bleaching agent(s).

As used herein, an enzyme is a protein (polypeptide) having catalytic activity.

As used herein, an “enzymatic bleaching system” or “enzymatic bleaching composition” includes one or more enzyme(s) and substrate(s) capable of enzymatically generating a bleaching agent. For example, an enzymatic bleaching system may contain a perhydrolase enzyme, an ester substrate, and a hydrogen peroxide source, for production of a peracid bleaching agent.

As used herein, an “ester substrate,” with reference to an enzymatic bleaching system containing a perhydrolase enzyme, refers to a perhydrolase substrate that contains an ester linkage. Esters comprising aliphatic and/or aromatic carboxylic acids and alcohols may be utilized as substrates with perhydrolase enzymes. In some embodiments, the ester source is an acetate ester. In some embodiments, the ester source is selected from one or more of propylene glycol diacetate, ethylene glycol diacetate, triacetin, ethyl acetate and tributyrin. In some embodiments, the ester source is selected from the esters of one or more of the following acids: formic acid, acetic acid, propionic acid, butyric acid, valeric acid, caproic acid, caprylic acid, nonanoic acid, decanoic acid, dodecanoic acid, myristic acid, palmitic acid, stearic acid, and oleic acid.

As used herein, the term “perhydrolase” refers to an enzyme that is capable of catalyzing a perhydrolysis reaction that results in the production of a sufficiently high amount of peracid suitable for use in a textile bleaching method as described. Generally, a perhydrolase enzyme exhibits a high perhydrolysis to hydrolysis ratio. In some embodiments, the perhydrolase comprises, consists of, or consists essentially of the Mycobacterium smegmatis perhydrolase amino acid sequence set forth in SEQ ID NO: 1, or a variant or homolog thereof. In some embodiments, the perhydrolase enzyme comprises acyl transferase activity and catalyzes an aqueous acyl transfer reaction.

As used herein, a “peracid” is an organic acid of the formula RC(═O)OOH.

As used herein, the term “hydrogen peroxide source” refers to hydrogen peroxide that is added to a textile treatment bath either from an exogenous (i.e., an external or outside) source or generated in situ by the action of a hydrogen peroxide generating oxidase on a substrate. A “hydrogen peroxide source” includes hydrogen peroxide as well as the components of a system that can spontaneously or enzymatically produce hydrogen peroxide as a reaction product.

The phrase “perhydrolysis to hydrolysis ratio” refers to the ratio of the amount of enzymatically produced peracid to the amount of enzymatically produced acid by a perhydrolase enzyme from an ester substrate under defined conditions and within a defined time. In some embodiments, the assays provided in WO 05/056782 are used to determine the amounts of peracid and acid produced by the enzyme.

As used herein, the term “acyl” refers to an organic group with the general formula RCO—, which can be derived from an organic acid by removal of the —OH group. Typically, acyl group names end with the suffix “-oyl,” e.g., methanoyl chloride, CH₃CO—Cl, is the acyl chloride formed from methanoic acid, CH₃CO—OH).

As used herein, the term “acylation” refers to a chemical transformation in which one of the substituents of a molecule is substituted by an acyl group, or the process of introduction of an acyl group into a molecule.

As used herein, the term “transferase” refers to an enzyme that catalyzes the transfer of a functional group from one substrate to another substrate. For example, an acyl transferase may transfer an acyl group from an ester substrate to a hydrogen peroxide substrate to form a peracid.

As used herein, the term “hydrogen peroxide generating oxidase” means an enzyme that catalyzes an oxidation/reduction reaction involving molecular oxygen (O₂) as the electron acceptor. In such a reaction, oxygen is reduced to water (H₂O) or hydrogen peroxide (H₂O₂). An oxidase suitable for use herein is an oxidase that generates hydrogen peroxide (as opposed to water) on its substrate. An example of a hydrogen peroxide generating oxidase and its substrate suitable for use herein is glucose oxidase and glucose. Other oxidase enzymes that may be used for generation of hydrogen peroxide include alcohol oxidase, ethylene glycol oxidase, glycerol oxidase, amino acid oxidase, and the like. In some embodiments, the hydrogen peroxide generating oxidase is a carbohydrate oxidase.

As used herein, the term “textile” refers to fibers, yarns, fabrics, garments, and non-wovens. The term encompasses textiles made from natural, synthetic (e.g., manufactured), and various natural and synthetic blends. Thus, the term “textile(s)” refers to unprocessed and processed fibers, yarns, woven or knit fabrics, non-wovens, and garments. In some embodiments, a textile contains cellulose.

As used herein, the phrase “textile(s) in need of processing” refers to textiles that need to be desized and/or scoured and/or bleached or may be in need of other treatments such as biopolishing.

As used herein, the phrase “textile(s) in need of bleaching” refers to textiles that need to be bleached without reference to other possible treatments. These textiles may or may not have been already subjected to other treatments. Similarly, these textiles may or may not need subsequent treatments.

As used herein, the term “fabric” refers to a manufactured assembly of fibers and/or yarns that has substantial surface area in relation to its thickness and sufficient cohesion to give the assembly useful mechanical strength.

As used herein, the phrase “effective amount of perhydrolase enzyme” refers to the quantity of perhydrolase enzyme necessary to achieve/produce the enzymatic activity required in the subject processes or methods. Such effective amounts are readily ascertained by one of ordinary skill in the art, and are based on many factors, such as the particular enzyme variant used, the pH used, the temperature used and the like, as well as the results desired (e.g., level of whiteness).

As used herein, the term “oxidizing chemical” refers to a chemical that has the ability to bleach a textile. The oxidizing chemical is present at an amount, pH, and temperature suitable for bleaching. The term includes, but is not limited to hydrogen peroxide and peracids.

As used herein, “oxidative stability” refers to the ability of a protein to function under oxidative conditions. In particular, the term refers to the ability of a protein to function in the presence of various concentrations of H₂O₂ and/or peracid. Stability under various oxidative conditions can be measured either by standard procedures known to those in the art. A substantial change in oxidative stability is evidenced by at least about a 5% or greater increase or decrease (in most embodiments, it is preferably an increase) in the half-life of the enzymatic activity, as compared to the enzymatic activity present in the absence of oxidative compounds.

As used herein, the term “pH stability,” with respect to a protein, refers to the ability of a protein to function and/or remain active at a particular pH. In general, most enzymes have a finite pH range at which they will function, and are stable. In addition to enzymes that function in mid-range pHs (i.e., around pH 7), there are enzymes that are capable of working under conditions with very high or very low pHs. Stability at various pHs can be measured either by standard procedures known to those in the art. A substantial change in pH stability is evidenced by at least about 5% or greater increase or decrease (in most embodiments, it is preferably an increase) in the half-life of the enzymatic activity, as compared to the enzymatic activity at the enzyme's optimum pH. However, it is not intended that the present processes, methods and/or compositions described herein be limited to any pH stability level nor pH range.

As used herein, “thermal stability,” with respect to a protein, refers to the ability of a protein to function and/or remain active at a particular temperature. In general, most enzymes have a finite range of temperatures at which they will function and remain active. In addition to enzymes that work in mid-range temperatures (e.g., room temperature), there are enzymes that are capable of working in very high or very low temperatures. Thermal stability can be measured either by known procedures. A substantial change in thermal stability is evidenced by at least about 5% or greater increase or decrease in the half-life of the catalytic activity of a mutant when exposed to a different temperature (i.e., higher or lower) than optimum temperature for enzymatic activity. However, it is not intended that the processes, methods and/or compositions described herein be limited to any temperature stability level nor temperature range.

As used herein, the term “chemical stability,” with respect to a protein, refers to the stability of a protein (e.g., an enzyme) towards chemicals that adversely affect its activity. In some embodiments, such chemicals include, but are not limited to hydrogen peroxide, peracids, anionic surfactants, cationic surfactants, non-ionic surfactants, chelants, and the like. However, it is not intended that the processes, methods and/or compositions described herein be limited to any particular chemical stability level nor range of chemical stability.

As used herein, the terms “purified” and “isolated” refer to the removal of contaminants from a sample and/or to a material (e.g., a protein, nucleic acid, cell, etc.) that is removed from at least one component with which it is naturally associated. For example, these terms may refer to a material which is substantially or essentially free from components which normally accompany it as found in its native state, such as, for example, an intact biological system

As used herein, the term “polynucleotide” refers to a polymeric form of nucleotides of any length and any three-dimensional structure and single- or multi-stranded (e.g., single-stranded, double-stranded, triple-helical, and the like), which contain deoxyribonucleotides, ribonucleotides, and/or analogs or modified forms of deoxyribonucleotides or ribonucleotides, including modified nucleotides or bases or their analogs. Because the genetic code is degenerate, more than one codon may be used to encode a particular amino acid, and the present compositions and methods encompasses polynucleotides which encode a particular amino acid sequence. Any type of modified nucleotide or nucleotide analog may be used, so long as the polynucleotide retains the desired functionality under conditions of use, including modifications that increase nuclease resistance (e.g., deoxy, 2′—O-Me, phosphorothioates, etc.). Labels may also be incorporated for purposes of detection or capture, for example, radioactive or nonradioactive labels or anchors, e.g., biotin. The term polynucleotide also includes peptide nucleic acids (PNA). Polynucleotides may be naturally occurring or non-naturally occurring. The terms “polynucleotide” and “nucleic acid” and “oligonucleotide” are used interchangeably. Polynucleotides may contain RNA, DNA, or both, and/or modified forms and/or analogs thereof. A sequence of nucleotides may be interrupted by non-nucleotide components. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. Polynucleotides may be linear or circular or comprise a combination of linear and circular portions.

As used herein, the term “polypeptide” refers to any composition comprised of amino acids and recognized as a protein by those of skill in the art. The conventional one-letter or three-letter codes for amino acid residues are used. The terms “polypeptide” and “protein” are used interchangeably to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, and the like.), as well as other modifications known in the art.

As used herein, the term “related proteins” refers to functionally and/or structurally similar proteins. In some embodiments, these proteins are derived from a different genus and/or species, including differences between classes of organisms (e.g., a bacterial protein and a fungal protein). In additional embodiments, related proteins are provided from the same species. Indeed, it is not intended that the processes, methods and/or compositions described herein be limited to related proteins from any particular source(s). In addition, the term “related proteins” encompasses tertiary structural homologs and primary sequence homologs. In further embodiments, the term encompasses proteins that are immunologically cross-reactive.

As used herein, the term “derivative” refers to a protein which is derived from a protein by addition of one or more amino acids to either or both the C- and N-terminal end(s), substitution of one or more amino acids at one or a number of different sites in the amino acid sequence, and/or deletion of one or more amino acids at either or both ends of the protein or at one or more sites in the amino acid sequence, and/or insertion of one or more amino acids at one or more sites in the amino acid sequence. The preparation of a protein derivative is preferably achieved by modifying a DNA sequence which encodes for the native protein, transformation of that DNA sequence into a suitable host, and expression of the modified DNA sequence to form the derivative protein.

As used herein, the term “variant proteins” refers to related and derivative proteins. In some embodiments, a variant proteins differ from a parent (or parental) protein, e.g., a wild-type protein, by the presence of different amino acid residues at a small number of amino acid positions. The number of different amino acid residues may be one or more, for example, 1, 2, 3, 4, 5, 10, 15, 20, 30, 40, 50, or more amino acid residues. The number of different amino acids may be between 1 and 10. Variant proteins may have defined level of sequence identity to a reference protein (for example, the parental protein), such as at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity. Alternatively or additionally, a variant protein may differ from a reference or parental protein in the number of prominent regions (i.e., domains, epitopes, or similar structural or functional portions). For example, in some embodiments, variant proteins have 1, 2, 3, 4, 5, or 10 corresponding prominent regions that differ from the parent protein. Methods known in the art are suitable for generating variants of the enzymes described herein, including but not limited to site-saturation mutagenesis, scanning mutagenesis, insertional mutagenesis, random mutagenesis, site-directed mutagenesis, and directed-evolution, as well as various other recombinant and combinatorial approaches.

As used herein, the term “analogous sequence” refers to a sequence within a protein that provides similar function, tertiary structure, and/or conserved residues as a reference protein (e.g., a protein of interest having a desirable structure or function). For example, in epitope regions that contain an alpha-helix or a beta-sheet structure, the replacement amino acids in the analogous sequence preferably maintain the same specific structure. The term also refers to nucleotide sequences, as well as amino acid sequences. In some embodiments, analogous sequences are developed such that the replacement amino acids result in a variant enzyme showing a similar or improved function. In some embodiments, the tertiary structure and/or conserved residues of the amino acids in the protein of interest are located at or near the segment or fragment of interest. Thus, where the segment or fragment of interest contains, for example, an alpha-helix or a beta-sheet structure, the replacement amino acids preferably maintain that specific structure.

As used herein, the term “homologous protein” refers to a protein (e.g., perhydrolase) that has similar action and/or structure, as a reference protein (e.g., a protein of interest, such as a perhydrolase, from another source). It is not intended that homologs be necessarily related evolutionarily. Thus, it is intended that the term encompass the same or similar enzyme(s) (i.e., in terms of structure and function) obtained from different species. In some embodiments, it is desirable to identify a homolog that has a quaternary, tertiary and/or primary structure similar to the protein of interest, as replacement for the segment or fragment in the protein of interest with an analogous segment from the homolog will reduce the disruptiveness of the change. In some embodiments, homologous proteins induce similar immunological response(s) as a protein of interest. In some embodiments, homologous proteins are engineered to produce enzymes with desired activity(ies).

As used herein, the terms “wild-type” and “native,” with respect to proteins and nucleic acids, refer to those found in nature. The terms “wild-type sequence,” and “wild-type gene” are used interchangeably herein, to refer to a sequence (protein or nucleic acid) that is native or naturally occurring in a host cell. In some embodiments, the wild-type sequence refers to a sequence of interest that is the starting point of a protein engineering project. The genes encoding the naturally-occurring protein may be obtained in accord with the general methods known to those skilled in the art. The methods generally comprise synthesizing labeled probes having putative sequences encoding regions of the protein of interest, preparing genomic libraries from organisms expressing the protein, and screening the libraries for the gene of interest by hybridization to the probes. Positively hybridizing clones are then mapped and sequenced.

The degree of homology between sequences may be determined using any suitable method known in the art (see, e.g., Smith and Waterman (1981) Adv. Appl. Math. 2:482; Needleman and Wunsch (1970) J. Mol. Biol. 48:443; Pearson and Lipman (1988) Proc. Natl. Acad. Sci. USA 85:2444; programs such as GAP, BESTFIT, FASTA, and TFASTA in the Wisconsin Genetics Software Package (Genetics Computer Group, Madison, Wis.); and Devereux et al. (1984) Nucleic Acids Res. 12:387-95).

For example, PILEUP is a useful program to determine sequence homology levels. PILEUP creates a multiple sequence alignment from a group of related sequences using progressive, pairwise alignments. It can also plot a tree showing the clustering relationships used to create the alignment. PILEUP uses a simplification of the progressive alignment method of Feng and Doolittle, (Feng and Doolittle (1987) J. Mol. Evol. 35:351-60). The method is similar to that described by Higgins and Sharp (Higgins and Sharp (1989) CABIOS 5:151-53). Useful PILEUP parameters including a default gap weight of 3.00, a default gap length weight of 0.10, and weighted end gaps. Another example of a useful algorithm is the BLAST algorithm, described by Altschul et al., (Altschul et al. (1990) J. Mol. Biol., 215:403-10; and Karlin et al. (1993) Proc. Natl. Acad. Sci. USA 90:5873-87). One particularly useful BLAST program is the WU-BLAST-2 program (Altschul et al. (1996) Meth. Enzymol. 266:460-80). Parameters “W,” “T,” and “X” determine the sensitivity and speed of the alignment. The BLAST program uses as defaults a wordlength (W) of 11, the BLOSUM62 scoring matrix (Henikoff and Henikoff (1989) Proc. Natl. Acad. Sci. USA 89:10915) alignments (B) of 50, expectation (E) of 10, M′S, N′−4, and a comparison of both strands.

As used herein, the phrases “substantially similar” and “substantially identical,” in the context of at least two nucleic acids or polypeptides, typically means that a polynucleotide or polypeptide comprises a sequence that has at least about 40% identity, at least about 50% identity, at least about 60% identity, at least about 75% identity, at least about 80% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity, compared to the reference (i.e., wild-type) sequence. Sequence identity may be determined using known programs such as BLAST, ALIGN, and CLUSTAL using standard parameters. (See, e.g., Altschul, et al. (1990) J. Mol. Biol. 215:403-10; Henikoff et al. (1989) Proc. Natl. Acad. Sci. USA 89:10915; Karin et al. (1993) Proc. Natl. Acad. Sci USA 90:5873; and Higgins et al. (1988) Gene 73:237 -44). Software for performing BLAST analyses is publicly available through the National Center for Biotechnology Information. Also, databases may be searched using FASTA (Pearson et al. (1988) Proc. Natl. Acad. Sci. USA 85:2444-48). One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, the terms “size” or “sizing” refer to compounds used in the textile industry to improve weaving performance by increasing the abrasion resistance and strength of the yarn. Size is usually made of, for example, starch or starch-like compounds.

As used herein, the terms “desize” or “desizing,” refer to the process of eliminating size, generally starch, from textiles usually prior to applying special finishes, dyes or bleaches.

As used herein, the term “desizing enzyme(s)” refers to enzymes that are used to enzymatically remove size. Exemplary enzymes are amylases, cellulases, and mannanases.

As used herein, the terms “perhydrolyzation,” “perhydrolyze,” or “perhydrolysis,” refer to a reaction wherein a peracid is generated from ester and hydrogen peroxide substrates. In one embodiment, the perhydrolyzation reaction is catalyzed with a perhydrolase, e.g., acyl transferase or aryl esterase, enzyme. In some embodiments, a peracid is produced by perhydrolysis of an ester substrate of the formula R₁C(═O)OR₂, where R₁ and R₂ are the same or different organic moieties, in the presence of hydrogen peroxide (H₂O₂). In one embodiment, —OR₂ is —OH. In one embodiment, —OR₂ is replaced by —NH₂. In some embodiments, a peracid is produced by perhydrolysis of a carboxylic acid or amide substrate.

As used herein, the term “peracid,” refers to a molecule derived from a carboxylic acid ester which has been reacted with hydrogen peroxide to form a highly reactive product that is able to transfer one of its oxygen atoms. It is this ability to transfer oxygen atoms that enables a peracid, for example, peracetic acid, to function as a bleaching agent.

As used herein, the term “scouring,” refers to removing impurities, for example, much of the non-cellulosic compounds (e.g., pectins, proteins, wax, motes, etc.) that are naturally found in cotton or other textiles. In addition to the natural non-cellulosic impurities, scouring can remove residual materials introduced by manufacturing processes, such as spinning, coning, or slashing lubricants. In some embodiments, bleaching may be employed to remove impurities from textiles.

As used herein, the term “bioscouring enzyme(s)” refers to an enzyme(s) capable of removing at least a portion of the impurities found in cotton or other textiles.

As used herein, the term “motes” refers to unwanted impurities, such as cotton seed fragments, leaves, stems, and other plant parts, which cling to the fiber even after a mechanical ginning process.

As used herein, the term “greige” (pronounced gray) refers to textiles that have not received any bleaching, dyeing, or finishing treatment after being produced. For example, any woven or knit fabric off the loom that has not yet been finished (desized, scoured, and the like), bleached, or dyed, is termed a greige textile. The textiles used in the examples, infra, are greige textiles.

As used herein, the term “dyeing,” refers to applying a color, e.g., to textiles, especially by soaking in a coloring solution.

As used herein, the term “non-cotton cellulosic” fiber, yarn, or fabric means fibers, yarns, or fabrics which are comprised primarily of a cellulose based composition other than cotton. Examples of such compositions include linen, ramie, jute, flax, rayon, lyocell, cellulose acetate, bamboo and other similar compositions which are derived from non-cotton cellulosics.

As used herein, the term “pectate lyase” refers to a type of pectinase. Pectinases are a group of enzymes that cleave glycosidic linkages of pectic substances mainly poly(1,4-alpha-D-galacturonide) and its derivatives (see Sakai et al. (1993) Advances in Applied Microbiology 39:213-294). Preferably, the pectinase catalyzes the random cleavage of alpha-1,4-glycosidic linkages in pectic acid (also called polygalacturonic acid) by transelimination, such as enzymes in the class polygalacturonate lyase (PGL; EC 4.2.2.2), also known as poly(1,4-alpha-D-galacturonide) lyase or pectate lyase.

As used herein, the term “pectin” denotes pectate, polygalacturonic acid and pectin, which may be esterified to a higher or lower degree.

As used herein, the term “cutinase,” refers to as a plant, bacterial or fungal derived lipolytic enzyme used in textile processing. Cutinases are capable of hydrolyzing the substrate, cutin. Cutinases can break down fatty acid esters and other oil-based compositions that need to be removed during textile processing (e.g., the scouring). In some embodiments, the cutinases has significant plant cutin hydrolysis activity. In particular embodiments, the cutinase has hydrolytic activity on the biopolyester polymer cutin found on the leaves of plants. Suitable cutinases may be isolated from many different plant, fungal and bacterial sources.

As used herein, the term “α-amylase” refers to an enzyme that cleaves the α(1-4)glycosidic linkages of amylose to yield maltose molecules (disaccharides of α-glucose). Amylases are digestive enzymes found in saliva and are also produced by many plants. Amylases break down long-chain carbohydrates (such as starch) into smaller units. An “oxidative stable” α-amylase is an α-amylase that is resistive to degradation by oxidative means, when compared to non-oxidative stable α-amylase, especially when compared to the non-oxidative stable α-amylase form which the oxidative stable α-amylase was derived.

As used herein, the term “protease” refers to a protein capable of catalyzing the cleavage of a peptide bond.

As used herein, a “catalase” refers to an enzyme that catalyzes the decomposition of hydrogen peroxide to hydrogen and oxygen.

As used herein, the term “wicking” refers to the passage of liquids along or through a textile material or a textile element of a coated fabric, or along interstices formed by a textile element and a coating polymer of a coated fabric. Wicking involves a spontaneous transport of a liquid driven into a porous system by capillary forces.

As used herein, the phrase “degree of polymerization” refers to the number of repeating units in the individual macromolecules in a polymer. Degree of polymerization may be based on a mass (weight) or a number average.

As used herein, the terms “fastness” or “color fastness” refer to ability of a material to resist color change, i.e., to retain its original hue, especially without fading, running, or changing when wetted, washed, cleaned, or stored under normal conditions when exposed to light, heat, or other influences.

As used herein, the terms “handle” or “hand” refer to the quality of a textile material, e.g., fabric or yarn, assessed by the reaction obtained from the sense of touch. It is concerned with the judgment of, for example, roughness, smoothness, harshness, pliability, thickness, and other tactile parameters.

As used herein, the term “pilling” refers to the entangling of textile fibers during washing, dry cleaning, testing, or in wear to form balls or pills which protrude from the surface of a fabric, and which are of such density that light will not pass through them, so that, e.g., they cast a shadow. Pilling that occurs during normal wear may be simulated, for example, on a laboratory-testing machine by controlled rubbing against an elastomeric pad having specifically selected mechanical properties. The degree of pilling may be evaluated against standards on an arbitrary scale ranging from 5 (indicating no pilling) to 1 (indicating very severe pilling).

As used herein, the term “surfactant” refers to a substance that reduces surface tension of a liquid.

As used herein, the term “emulsifier” refers to a substance that promotes the suspension of one liquid in another.

As used herein, the term “sequestering agent” refers to a substance capable of reacting with metallic ions by forming a water-soluble complex in which the metal is held in a non-ionizable form.

As used herein, the terms “batch process,” “batchwise process,” or “discontinuous process” refer to the processing of textiles in lots or batches in which the entirety of each batch is subjected to a process or one stage of a process at a time.

As used herein, the term “exhaust process” refers to a batch process in which pretreatment chemicals and/or an enzymatic pretreatment composition and dye are added simultaneously or sequentially in a single textile treatment bath.

As used herein, the term “liquor ratio” refers to the ratio of the weight of liquor (liquid) employed in a textile treatment process to the weight of the textile treated.

### Enzymatic Textile Bleaching Compositions

One aspect of the compositions and methods provides enzymatic bleaching compositions and methods for bleaching textiles using these compositions. Textiles include cellulose-containing textiles, e.g., textiles made from cotton, flax, hemp, ramie, cellulose, acetate, lyocell, viscose rayon, bamboo, and various cellulosic blends, as well as textiles made from polyamide, polyacrylic, wool, or blends thereof. In some embodiments, the textile comprises a blend with elastane. The enzymatic bleaching compositions and methods are particularly useful for bleaching textiles containing fibers that are sensitive to high pH and temperature conditions. The enzymatic bleaching compositions and methods are particularly useful in batch, exhaust, or discontinuous processes.

The enzymatic bleaching compositions contain a perhydrolase enzyme, an ester substrate for the perhydrolase enzyme suitable for production of a peracid upon catalytic reaction of the perhydrolase enzyme on the substrate in the presence of hydrogen peroxide, a hydrogen peroxide source, a surfactant and/or an emulsifier, a peroxide stabilizer, a sequestering agent, and a buffer which maintains a pH of about 6 to about 8 during a textile bleaching process using the enzymatic bleaching composition. The enzymatic bleaching composition may optionally further contain a bioscouring agent or enzyme and/or a desizing agent or enzyme.

The enzymatic bleaching compositions, when used in a textile pretreatment process, advantageously produce bleached textiles that exhibit increased dye uptake, decreased textile damage due to the bleaching process, and/or a bulkier softer handle when compared to pretreatment with a chemical bleaching composition that does not contain the perhydrolase enzyme. In some embodiments, the enzymatic bleaching compositions, when used in a textile pretreatment process, produce textiles with reduced pilling propensity.

### Perhydrolase Enzyme

The enzymatic bleaching compositions include one or more perhydrolase enzymes. In some embodiments, the perhydrolase enzyme is naturally-occurring (i.e., a perhydrolase enzyme encoded by the genome of a cell). In some embodiments, the perhydrolase enzyme comprises, consists of, or consists essentially of an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or even at least about 99.5% identical to the amino acid sequence of a naturally-occurring perhydrolase enzyme.

In some embodiments, a perhydrolase enzyme is a naturally occurring M. smegmatis perhydrolase enzyme. In some embodiments, a perhydrolase enzyme comprises, consists of, or consists essentially of the amino acid sequence set forth in SEQ ID NO: 1 or a variant or homologue thereof. In some embodiments, a perhydrolase enzyme comprises, consists of, or consists essentially of an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or even at least about 99.5% identical to the amino acid sequence set forth in SEQ ID NO: 1.

The amino acid sequence of M. smegmatis perhydrolase (SEQ ID NO: 1) is:

MAKRILCFGDSLTWGWVPVEDGAPTERFAPDVRWTGVLAQQLGADFEVI
EEGLSARTTNIDDPTDPRLNGASYLPSCLATHLPLDLVIIMLGTNDTKA
YFRRTPLDIALGMSVLVTQVLTSAGGVGTTYPAPKVLVVSPPPLAPMPH
PWFQLIFEGGEQKTTELARVYSALASFMKVPFFDAGSVISTDGVDGIHF
TEANNRDLGVALAEQVRSLL

The corresponding polynucleotide sequence encoding M. smegmatis perhydrolase (SEQ ID NO:2) is:

5′-ATGGCCAAGCGAATTCTGTGTTTCGGTGATTCCCTGACCTGGGGCT
GGGTCCCCGTCGAAGACGGGGCACCCACCGAGCGGTTCGCCCCCGACGT
GCGCTGGACCGGTGTGCTGGCCCAGCAGCTCGGAGCGGACTTCGAGGTG
ATCGAGGAGGGACTGAGCGCGCGCACCACCAACATCGACGACCCCACCG
ATCCGCGGCTCAACGGCGCGAGCTACCTGCCGTCGTGCCTCGCGACGCA
CCTGCCGCTCGACCTGGTGATCATCATGCTGGGCACCAACGACACCAAG
GCCTACTTCCGGCGCACCCCGCTCGACATCGCGCTGGGCATGTCGGTGC
TCGTCACGCAGGTGCTCACCAGCGCGGGCGGCGTCGGCACCACGTACCC
GGCACCCAAGGTGCTGGTGGTCTCGCCGCCACCGCTGGCGCCCATGCCG
CACCCCTGGTTCCAGTTGATCTTCGAGGGCGGCGAGCAGAAGACCACTG
AGCTCGCCCGCGTGTACAGCGCGCTCGCGTCGTTCATGAAGGTGCCGTT
CTTCGACGCGGGTTCGGTGATCAGCACCGACGGCGTCGACGGAATCCAC
TTCACCGAGGCCAACAATCGCGATCTCGGGGTGGCCCTCGCGGAACAGG
TGCGGAGCCTGCTGTAA-3′

In some embodiments, the perhydrolase enzyme comprises one or more substitutions at one or more amino acid positions equivalent to position(s) in the M. smegmatis perhydrolase amino acid sequence set forth in SEQ ID NO: 1. In some embodiments, the perhydrolase enzyme comprises any one or any combination of substitutions of amino acids selected from M1, K3, R4, I5, L6, C7, D10, S11, L12, T13, W14, W16, G15, V17, P18, V19, D21, G22, A23, P24, T25, E26, R27, F28, A29, P30, D31, V32, R33, W34, T35, G36, L38, Q40, Q41, D45, L42, G43, A44, F46, E47, V48, I49, E50, E51, G52, L53, S54, A55, R56, T57, T58, N59, I60, D61, D62, P63, T64, D65, P66, R67, L68, N69, G70, A71, S72, Y73, S76, C77, L78, A79, T80, L82, P83, L84, D85, L86, V87, N94, D95, T96, K97, Y99F100, R101, R102, P104, L105, D106, I107, A108, L109, G110, M111, S112, V113, L114, V115, T116, Q117, V118, L119, T120, S121, A122, G124, V125, G126, T127, T128, Y129,P146, P148, W149, F150, I153, F154, I194, and F196.

In some embodiments, the perhydrolase enzyme comprises one or more of the following substitutions at one or more amino acid positions equivalent to position(s) in the M. smegmatis perhydrolase amino acid sequence set forth in SEQ ID NO: 1: L12C, Q, or G; T25S, G, or P; L53H, Q, G, or S; S54V, L A, P, T, or R; A55G or T; R67T, Q, N, G, E, L, or F; K97R; V125S, G, R, A, or P; F154Y; F196G.

In some embodiments, the perhydrolase enzyme comprises a combination of amino acid substitutions at amino acid positions equivalent to amino acid positions in the M. smegmatis perhydrolase amino acid sequence set forth in SEQ ID NO: 1: L12I S54V; L12M S54T; L12T S54V; L12Q T25S S54V; L53H S54V; S54P V125R; S54V V125G; S54V F196G; S54V K97R V125G; or A55G R67T K97R V125G.

In some embodiments, the perhydrolase enzyme has a perhydrolysis:hydrolysis ratio of at least 1. In some embodiments, the perhydrolase enzyme has a perhydrolysis:hydrolysis ratio greater than 1.

In some embodiments, the perhydrolase enzyme is provided in the enzymatic bleaching composition at a concentration of about 1 to about 2.5 pm, about 1.5 to about 2.0 ppm, or about 1.7 ppm.

### Ester Substrate

The present enzymatic bleaching compositions further include an ester, which serves as a substrate for the perhydrolase enzyme for production of a peracid in the presence of hydrogen peroxide. In some embodiments, the ester substrate is an ester of an aliphatic and/or aromatic carboxylic acid or alcohol. In some embodiments, the ester substrate is an ester of one or more of the following: formic acid, acetic acid, propionic acid, butyric acid, valeric acid, caproic acid, caprylic acid, nonanoic acid, decanoic acid, dodecanoic acid, myristic acid, palmitic acid, stearic acid, and oleic acid. In some embodiments, triacetin, tributyrin, and other esters serve as acyl donors for peracid formation. In some embodiments, the ester substrate is propylene glycol diacetate, ethylene glycol diacetate, or ethyl acetate. In one embodiment, the ester substrate is propylene glycol diacetate.

In some embodiments, the ester substrate is provided at a concentration of about 2,000 to about 4,000 ppm, about 2,500 to about 3,500 ppm, about 2,800 ppm to about 3,200 ppm, or about 3,000 ppm.

### Hydrogen Peroxide Source

The present enzymatic bleaching compositions further include a hydrogen peroxide source. Hydrogen peroxide can be either added directly in batch, or generated continuously “in situ” by chemical, electro-chemical, and/or enzymatic means.

In some embodiments, the hydrogen peroxide source is hydrogen peroxide. In some embodiments, the hydrogen peroxide source is a solid compound that generates hydrogen peroxide upon addition to water. Such compounds include adducts of hydrogen peroxide with various inorganic or organic compounds, of which the most widely employed is sodium carbonate per hydrate, also referred to as sodium percarbonate.

Inorganic perhydrate salts are one preferred embodiment of hydrogen peroxide source. Examples of inorganic perhydrate salts include perborate, percarbonate, perphosphate, persulfate, and persilicate salts. The inorganic perhydrate salts are normally the alkali metal salts.

Other hydrogen peroxide adducts useful in the present compositions include adducts of hydrogen peroxide with zeolites, or urea hydrogen peroxide.

The hydrogen peroxide source compounds may be included as a crystalline and/or substantially pure solid without additional protection. However, for certain granular perhydrate salts, the preferred forms are coated with a material that provides better storage stability. Suitable coatings include inorganic salts such as alkali metal silicate, carbonate or borate salts or mixtures thereof, or organic materials such as waxes, oils, or fatty soaps.

In some embodiments, the hydrogen peroxide source is an enzymatic hydrogen peroxide generation system. In one embodiment, the enzymatic hydrogen peroxide generation system comprises an oxidase and its substrate. Suitable oxidase enzymes include, but are not limited to: glucose oxidase, sorbitol oxidase, hexose oxidase, choline oxidase, alcohol oxidase, glycerol oxidase, cholesterol oxidase, pyranose oxidase, carboxyalcohol oxidase, L-amino acid oxidase, glycine oxidase, pyruvate oxidase, glutamate oxidase, sarcosine oxidase, lysine oxidase, lactate oxidase, vanillyl oxidase, glycolate oxidase, galactose oxidase, uricase, oxalate oxidase, and xanthine oxidase.

The following equation provides an example of a coupled system for enzymatic production of hydrogen peroxide.

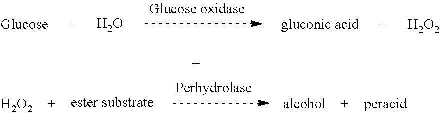

It is not intended that the present compositions and methods be limited to any specific enzyme, as any enzyme that generates H₂O₂ with a suitable substrate may be used. For example, lactate oxidases from Lactobacillus species which are known to create H₂O₂ from lactic acid and oxygen may be used. One advantage of the enzymatic generation of acid (e.g., gluconic acid in the above example) is that this reduces the pH of a basic solution to the pH range in which a peracid is most effective in bleaching (i.e., at or below the pKa). Other enzymes (e.g., alcohol oxidase, ethylene glycol oxidase, glycerol oxidase, amino acid oxidase, and the like) that can generate hydrogen peroxide may also be used in combination with perhydrolase enzymes and ester substrates to generate peracids.

Hydrogen peroxide may also be generated electrochemically, for example using a fuel cell fed oxygen and hydrogen gas.

In some embodiments, the hydrogen peroxide source is hydrogen peroxide provided at a concentration of about 1,000 to about 3,000 ppm, about 1,500 to about 2,500 ppm, about 2,000 ppm to about 2,200 ppm, or about 2,100 ppm.

### Surfactants and Emulsifiers

The present enzymatic textile bleaching compositions may further include one or more, i.e., at least one, surfactant(s) and/or emulsifier(s). Suitable surfactants include, without limitation, nonionic (see, e.g., U.S. Pat. No. 4,565,647, which is herein incorporated by reference); anionic; cationic; and zwitterionic surfactants (see, e.g., U.S. Pat. No. 3,929,678). Anionic surfactants include, without limitation, linear alkylbenzenesulfonate, α-olefinsulfonate, alkyl sulfate (fatty alcohol sulfate), alcohol ethoxysulfate, secondary alkanesulfonate, α-sulfo fatty acid methyl ester, alkyl- or alkenylsuccinic acid, and soap. Non-ionic surfactants include, without limitation, alcohol ethoxylate, nonylphenol ethoxylate, alkylpolyglycoside, alkyldimethylamineoxide, ethoxylated fatty acid monoethanolamide, fatty acid monoethanolamide, polyhydroxy alkyl fatty acid amide, and N-acyl N-alkyl derivatives of glucosamine (“glucamides”).

In some embodiments, the enzymatic bleaching composition contains a non-ionic surfactant. In one embodiment, the non-ionic surfactant is an alcohol ethoxylate.

A surfactant may be present at a concentration of about 5% to about 40%, about 20% to about 30%, or about 5% to about 10% (w/w).

In some embodiments, the enzymatic bleaching composition contains ethoxylated isotridecanol at a concentration of about 5% to about 30%, about 10% to about 25%, or about 15% to about 20% (w/w).

### Peroxide Stabilizers

The present enzymatic bleaching compositions may further include a peroxide stabilizer. Examples of peroxide stabilizers include, but are not limited to, sodium silicate, sodium carbonate, acrylic polymers, magnesium salts, and phosphonic acid. In one embodiment, the peroxide stabilizer is phosphonic acid.

The peroxide stabilizer may be present in an enzymatic bleaching composition at a concentration of about 1% to about 5%, about 1% to about 10%, or about 2% to about 8% (w/w).

### Sequestering Agents

The present enzymatic bleaching compositions may further include a sequestering agent. Examples of sequestering agents include, but are not limited to, amino carboxylates, amino phosphonates, polyfunctionally-substituted aromatic chelating agents, polyhydroxy-carboxylic acids, aminopolycarboxylic acids, polyphosphonates, and polyacrylic acids, and mixtures thereof. Particular amino carboxylates useful as sequestering agents include ethylenediaminetetracetates, N-hydroxyethylethylenediaminetriacetates, nitrilotriacetates, ethylenediamine tetraproprionates, and triethylenetetraaminehexacetates.

Polyfunctionally-substituted aromatic sequestering agents are also useful in the present compositions (see, e.g., U.S. Pat. No. 3,812,044, issued May 21, 1974, to Connor et al.). Preferred compounds of this type in acid form are dihydroxydisulfobenzenes such as 1,2-dihydroxy-3,5-disulfobenzenediethylenetriaminepentaacetates, and ethanoldiglycines, alkali metal, ammonium, and substituted ammonium salts therein and mixtures thereof.

Amino phosphonates are also suitable for use as sequestering agents in the present compositions, particularly when at least low levels of total phosphorus are permitted.

A biodegradable sequestering agent suitable for use herein is ethylenediamine disuccinate (“EDDS”), especially the [S,S] isomer as described in U.S. Pat. No. 4,704,233 (issued Nov. 3, 1987 to Hartman and Perkins).

In one embodiment, the sequestering agent is polyacrylic acid.

A sequestering agent may be present in an enzymatic bleaching composition described herein at a concentration of about 1% to about 15%, about 5% to about 10%, or about 3% to about 10% (w/w).

### Buffers

The present enzymatic bleaching compositions may include a buffer that is capable of maintaining the pH of the composition at a pH of about 6 to about 8. In one embodiment, the buffer is a phosphate buffer, for example, 100 mM phosphate buffer, pH 8.

### Enzymatic Textile Bleaching Methods

Another aspect of the compositions and methods provides methods for bleaching of textiles, using any of the enzymatic bleaching compositions described herein. Generally, the textile to be bleached is contacted with an enzymatic textile composition as described herein for a length of time and under conditions suitable to permit measurable whitening of the textile.

Textiles include cellulose-containing textiles, e.g., textiles made from cotton, flax, hemp, ramie, cellulose, acetate, lyocell, viscose rayon, bamboo, and various cellulosic blends, as well as textiles made from polyamide, polyacrylic, wool, or blends thereof. In some embodiments, the textile comprises a blend with elastane. The enzymatic bleaching compositions and methods are particularly useful for bleaching textiles containing fibers that are sensitive to high pH and temperature conditions.

Advantageously, treatment of textiles in accordance with the methods produces bleached textiles with increased dye uptake, decreased textile damage, and/or bulkier softer handle when compared to a chemical bleaching process using a chemical bleaching composition that does not include a perhydrolase enzyme. In some embodiments, textiles are produced that have a reduced pilling process when compared with a chemical bleaching process that does not include a perhydrolase enzyme.

The present enzymatic bleaching further advantageously require less energy due to the lower processing temperatures that are employed in comparison to a typical chemical bleaching process. In addition, less rinsing is required than a chemical bleaching process, resulting in lower water usage. The present methods also produces a lower pH effluent (<8) than chemical bleaching (about 13), resulting in reduced adverse environmental impact.

Typically, the present methods utilize a liquor ratio of about 6:1 to about 15:1, for example, about 10:1. In some embodiments, the methods are performed in a batch, exhaust, or discontinuous textile bleaching process.

Textiles are contacted with the enzymatic bleaching composition at a temperature of about 40° to about 70° C., for example, about 60° C. to about 70° C., for a processing time about 40 to about 60 minutes. In one embodiment, the bleaching temperature is about 65° C. and the processing time is about 50 minutes. In some embodiments, the temperature of the enzymatic bleaching composition is raised by about 3° C. per minute from a starting temperature of about 20° C. to about 50° C. until the processing temperature for bleaching is reached.

In some embodiments, one or more rinsing steps are performed after incubation of the textile in the enzymatic bleaching composition, to remove the bleaching composition. Typically, the textile is rinsed with an aqueous composition (water or a composition containing water). In some embodiments, the rinsing temperature is about 40° C. to about 60° C., for example, about 50° C. In some embodiments, the aqueous rinsing composition contains a catalase enzyme to hydrolyze the hydrogen peroxide. In one embodiment, the textile is rinsed twice with a catalase containing aqueous composition for about 10 minutes for each rinse.

In some embodiments, textiles bleached using the methods herein contain a softer, bulkier, and more natural handle than textiles bleached propensity when compared to a textile treated with a chemical bleaching composition that does not comprise a perhydrolase enzyme. This bulkier, softer handle often results in an improvement in sewability (needle resistance) and stretch. Further, the permanent bulkier, softer handle often results in improvement in crease recovery, e.g., lower risk for crease marking in piece good and garment processing.

In some embodiments, properties of elastane are enhanced using the enzymatic bleaching methods herein, in comparison to bleaching with a chemical process that does not comprise a perhydrolase enzyme.

In some embodiments, the enzymatic bleaching methods herein result in natural fibers with less swelling and avoidance of channeling effect in yarn cheese dyeing machines, in comparison to a chemical bleaching process that does not comprise a perhydrolase enzyme.

### Bioscouring Enzymes

In some embodiments, the present compositions and methods for enzymatic textile bleaching include one or more bioscouring enzymes. The bioscouring enzyme(s) may be included in the enzymatic textile bleaching composition, or a textile may be treated with the bioscouring enzyme(s) in a subsequent processing step after pretreatment in the enzymatic textile bleaching composition. Exemplary bioscouring enzymes are described, below.

Pectinases

Any pectinolytic enzyme having the ability to degrade the pectin component of, e.g., plant cell walls, may be used in the present compositions and methods. Suitable pectinases include, without limitation, those of fungal or bacterial origin. The pectinases may be of natural origin or recombinantly produced, and/or may be chemically or genetically modified. In some embodiments, the pectinases are mono-component enzymes.

Pectinases can be classified according to their preferential substrate, highly methyl-esterified pectin or low methyl-esterified pectin and polygalacturonic acid (pectate), and their reaction mechanism, β-elimination or hydrolysis. Pectinases can be mainly endo-acting, cutting the polymer at random sites within the chain to give a mixture of oligomers, or they may be exo-acting, attacking from one end of the polymer and producing monomers or dimers. Several pectinase activities acting on the smooth regions of pectin are included in the classification of enzymes provided by Enzyme Nomenclature (1992), e.g., pectate lyase (EC 4.2.2.2), pectin lyase (EC 4.2.2.10), polygalacturonase (EC 3.2.1.15), exo-polygalacturonase (EC 3.2.1.67), exo-polygalacturonate-lyase (EC 4.2.2.9) and exo-poly-alpha-galacturonosidase (EC 3.2.1.82). In preferred embodiments, the methods utilize pectate lyases.

Pectate lyase enzymatic activity as used herein refers to catalysis of the random cleavage of α-1,4-glycosidic linkages in pectic acid (also called polygalacturonic acid) by transelimination. Pectate lyases are also termed polygalacturonate lyases and poly(1,4-D-galacturonide) lyases. For purposes of the present compositions and methods, pectate lyase enzymatic activity is the activity determined by measuring the increase in absorbance at 235 nm of a 0.1% w/v solution of sodium polygalacturonate in 0.1 M glycine buffer at pH 10 (See Collmer et al. (1988) Methods Enzymol 161:329-35). Enzyme activity is typically expressed as x mol/min, i.e., the amount of enzyme that catalyzes the formation of x mole product/min. An alternative assay measures the decrease in viscosity of a 5% w/v solution of sodium polygalacturonate in 0.1 M glycine buffer at pH 10, as measured by vibration viscometry (APSU units). It will be understood that any pectate lyase may be used in practicing the present compositions and methods.

Non-limiting examples of pectate lyases whose use is encompassed by the present present compositions and methods include pectate lyases that have been cloned from different bacterial genera such as Erwinia, Pseudomonas, Bacillus, Klebsiella and Xanthomonas. Pectate lyases suitable for use herein are from Bacillus subtilis (Nasser et al. (1993) FEBS Letts. 335:319-26) and Bacillus sp. YA-14 (Kim et al. (1994) Biosci. Biotech. Biochem. 58:947-49). Other pectate lyases produced by Bacillus pumilus (Dave and Vaughn (1971) J. Bacteriol. 108:166-74), B. polymyxa (Nagel and Vaughn (1961) Arch. Biochem. Biophys. 93:344-52), B. stearothermophilus (Karbassi and Vaughn (1980) Can. J. Microbiol. 26:377-84), Bacillus sp. (Hasegawa and Nagel (1966) J. Food Sci. 31:838-45) and Bacillus sp. RK9 (Kelly and Fogarty (1978) Can. J. Microbiol. 24:1164-72) have also been described and are contemplated to be used in the present compositions and methods. Any of the above, as well as divalent cation-independent and/or thermostable pectate lyases, may be used in practicing the present compositions and methods. In some embodiments, the pectate lyase comprises, for example, those disclosed in WO 04/090099 (Diversa) or WO 03/095638 (Novozymes).

An effective amount of pectolytic enzyme to be used according to the method of the present compositions and methods depends on many factors, but according to the present compositions and methods the concentration of the pectolytic enzyme in the aqueous medium may be from about 0.0001% to about 1% μg enzyme protein by weight of the fabric, such as about 0.0005% to about 0.2% enzyme protein by weight of the fabric, or about 0.001% to about 0.05% enzyme protein by weight of the fabric.

Enzymes that Hydrolyze Polyester Substrates

Any enzyme that hydrolyzes a polyester substrate is suitable for use in the present compositions and methods, for example, a cutinase or lipase, including, for example, the enzyme derived from Humicola insolens strain DSM 1800, as described in Example 2 of U.S. Pat. No. 4,810,414 or, in one embodiment, the enzyme from Pseudomonas mendocina described in U.S. Pat. No. 5,512,203, variants thereof and/or equivalents. Suitable variants are described, for example, in WO 03/76580. These documents are incorporated herein by reference.

Suitable bacterial enzymes may be derived from a Pseudomonas or Acinetobacter species, preferably from P. stutzeri, P. alcaligenes, P. pseudoalcaligenes, P. aeruginosa or A. calcoaceticus, most preferably from P. stutzeri strain Thai IV 17-1 (CBS 461.85), PG-1-3 (CBS 137.89), PG-1-4 (CBS 138.89), PG-II-1 1.1 (CBS 139.89) or PG-II-1 1.2 (CBS 140.89), P. aeruginosa PAO (ATCC 15692), P. alcaligenes DSM 50342, P. pseudoalcaligenes IN II-5 (CBS 468.85), P. pseudoalcaligenes M-1 (CBS 473.85) or A. calcoaceticus Gr V-39 (CBS 460.85). With respect to the use of enzymes derived from plants, it is known that enzymes that hydrolyze polyester substrates exist in the pollen of many plants and such enzymes would be useful in the present processes, methods and compositions. Enzymes that hydrolyze polyester substrates may also be derived a from fungus, such as, Absidia spp.; Acremonium spp.; Agaricus spp.; Anaeromyces spp.; Aspergillus spp., including A. auculeatus, A. awamori, A. flavus, A. foetidus, A. fumaricus, A. fumigatus, A. nidulans, A. niger, A. oryzae, A. terreus and A. versicolor; Aeurobasidium spp.; Cephalosporum spp.; Chaetomium spp.; Coprinus spp.; Dactyllum spp.; Fusarium spp., including F. conglomerans, F. decemcellulare, F. javanicum, F. lini, F.oxysporum and F. solani; Gliocladium spp.; Humicola spp., including H. insolens and H. lanuginosa; Mucor spp.; Neurospora spp., including N. crassa and N. sitophila; Neocallimastix spp.; Orpinomyces spp.; Penicillium spp; Phanerochaete spp.; Phlebia spp.; Piromyces spp.; Pseudomonas spp.; Rhizopus spp.; Schizophyllum spp.; Trametes spp.; Trichoderma spp., including T. reesei, T. reesei (longibrachiatum) and T. viride; and Zygorhynchus spp. Similarly, it is envisioned that an enzyme that hydrolyzes a polyester substrate may be found in bacteria such as Bacillus spp.; Cellulomonas spp.; Clostridium spp.; Myceliophthora spp.; Pseudomonas spp., including P. mendocina and P. putida; Thermomonospora spp.; Thermomyces spp., including T. lanuginose; Streptomyces spp., including S. olivochromogenes; and in fiber degrading ruminal bacteria such as Fibrobacter succinogenes; and in yeast including Candida spp., including C. Antarctica, C. rugosa, C. torresii; C. parapsllosis; C. sake; C. zeylanoides; Pichia minuta; Rhodotorula glutinis; R. mucilaginosa; and Sporobolomyces holsaticus.

In some embodiments, enzymes that hydrolyze polyester substrates, for example, a cutinase and/or a lipase, are incorporated in the enzymatic bleaching composition in an amount from about 0.00001% to about 2% of enzyme protein by weight of the fabric, such as in an amount from about 0.0001% to about 1% of enzyme protein by weight of the fabric, or in an amount from 0.005% to 0.5% of enzyme protein by weight of the fabric, often in an amount from about 0.001% to about 0.5% of enzyme protein by weight of the fabric.

Cellulases

Cellulases may be added to the present compositions and methods, e.g., to promote bioscouring. Cellulases are classified as a series of enzyme families encompassing endo- and exo- activities as well as cellobiose hydrolyzing capability. The cellulase may be derived from microorganisms which are known to be capable of producing cellulolytic enzymes, such as, e.g., species of Humicola, Thermomyces, Bacillus, Trichoderma, Fusarium, Myceliophthora, Phanerochaete, Irpex, Scytalidium, Schizophyllum, Penicillium, Aspergillus or Geotricum. Known species capable for producing celluloytic enzymes include Humicola insolens, Fusarium oxysporum or Trichoderma reesei. Non-limiting examples of suitable cellulases are disclosed in U.S. Pat. No. 4,435,307; European patent application No. 0 495 257; PCT Patent Application No. WO91/17244; and European Patent Application No. EP-A2-271 004, all of which are incorporated herein by reference.

Cellulases are also useful for biopolishing of the textile. Cotton and other natural fibers based on cellulose can be improved by enzymatic biopolishing to produce a fabric with a smoother and glossier appearance. The treatment is used to remove “fuzz,” i.e., the tiny strands of fiber that protrude from the surface of yarn. A ball of fuzz is called a “pill” in the textile trade. After biopolishing, the fuzz and pilling are reduced. The other benefits of removing fuzz are a softer and smoother handle and superior color brightness.

In some embodiments of the present compositions and methods, the cellulase may be used at a concentration in the range from about 0.0001% to about 1% enzyme protein by weight of the fabric, such as about 0.0001% to about 0.05% enzyme protein by weight of the fabric, or about 0.0001 to about 0.01% enzyme protein by weight of the fabric.

In some embodiments, one or more cellulase enzyme is included in the enzymatic textile bleaching composition as described herein, and a system for removing hydrogen peroxide, e.g., catalase, is added after the bleached and biopolished textile is produced.

In some embodiments, a method for combined bleaching and biopolishing of a textile is provided, comprising (i) contacting the textile with an enzymatic bleaching composition as described herein and a biopolishing enzyme, e.g., a cellulase enzyme, for a length of time and under conditions suitable to permit measurable whitening of the textile and biopolishing of the textile, wherein the bleached and biopolished textile comprises at least one of decreased textile damage, bulkier softer handle, and increased dye uptake when compared to a chemical bleaching method that comprises contacting the textile with a chemical textile bleaching composition that does not comprise a perhydrolase enzyme; and (ii) hydrolyzing hydrogen peroxide with a system for removing hydrogen peroxide, e.g., a catalase enzyme, after the bleached and biopolished textile is produced.

Determination of cellulase activity (ECU) The cellulolytic activity may be determined in endo-cellulase units (ECU) by measuring the ability of the enzyme to reduce the viscosity of a solution of carboxymethyl cellulose (CMC), The ECU assay quantifies the amount of catalytic activity present in the sample by measuring the ability of the sample to reduce the viscosity of a solution of carboxy- methylcellulose (CMC). The assay is carried out in a vibration viscosimeter (e.g., MIVI 3000 from Sofraser, France) at 40° C.; pH 7.5; 0.1 M phosphate buffer; time 30 minutes using a relative enzyme standard for reducing the viscosity of the CHIC substrate (Hercules 7 LED), enzyme concentration approx. 0.15 ECU/ml. The arch standard is defined to 8,200 ECU/g. One ECU is amount of enzyme that reduces the viscosity to one half under these conditions.

Other Bioscouring Enzymes

The present compositions and methods are not limited to the use of the enzymes discussed above for bioscouring. Other enzymes may be used either alone or in combination with each other or with those listed above. For example, proteases may be used in the present compositions and methods. Suitable proteases include those of animal, vegetable or microbial origin, preferably of microbial origin. The protease may be a serine protease or a metalloprotease, preferably an alkaline microbial protease or a trypsin-like protease. Examples of proteases include aminopeptidases, including prolyl aminopeptidase (3.4.11.5), X-pro aminopeptidase (3.4.11.9), bacterial leucyl aminopeptidase (3.4.11.10), thermophilic aminopeptidase (3.4.11.12), lysyl aminopeptidase (3.4.11.15), tryptophanyl aminopeptidase (3.4.11.17), and methionyl aminopeptidase (3.4.11.18); serine endopeptidases, including chymotrypsin (3.4.21.1), trypsin (3.4.21.4), cucumisin (3.4.21.25), brachyurin (3.4.21.32), cerevisin (3.4.21.48)and subtilisin (3.4.21.62); cysteine endopeptidases, including papain (3.4.22.2), ficain (3.4.22.3), chymopapain (3.4.22.6), asclepain (3.4.22.7), actinidain (3.4.22.14), caricain (3.4.22.30) and ananain (3.4.22.31); aspartic endopeptidases, including pepsin A (3.4.23.1), Aspergillopepsin I (3.4.23.18), Penicillopepsin (3.4.23.20) and Saccharopepsin (3.4.23.25); and metalloendopeptidases, including Bacillolysin (3.4.24.28).

Non-limiting examples of subtilisins include subtilisin BPN′, subtilisin amylosacchariticus, subtilisin 168, subtilisin mesentericopeptidase, subtilisin Carlsberg, subtilisin DY, subtilisin 309, subtilisin 147, thermitase, aqualysin, Bacillus PB92 protease, proteinase K, protease TW7, and protease TW3.

Commercially available proteases include ALCALASE™, SAVINASE™, PRIMASE™, DURALASE™, ESPERASE™, KANNASE™, and DURAZYM™ (Novo Nordisk A/S), MAXATASE™, MAXACAL™, MAXAPEM™, PROPERASE™, Purafect™, PURAFECT OXP™, FN2.™ and FN3™ (Genencor Division, Danisco US Inc.).

Also useful in the present compositions and methods are protease variants, such as those disclosed in patents or published patent applications EP 130,756 (Genentech), EP 214,435 (Henkel), WO 87/04461 (Amgen), WO 87/05050 (Genex), EP 251,446 (Genencor), EP 260,105 (Genencor), Thomas et al. (1985) Nature 318:375-76, Thomas et al. (1987) J. Mol. Biol. 193:803-13, Russel et al. (1987) Nature 328:496-500, WO 88/08028 (Genex), WO 88/08033 (Amgen), WO 89/06279 (Novo Nordisk A/S), WO 91/00345 (Novo Nordisk A/S), EP 525 610 (Solvay) and WO 94/02618 (Gist-Brocades N.V.), all of which are incorporated herein by reference.

The activity of proteases can be determined as described in “Methods of Enzymatic Analysis,” third edition, 1984, Verlag Chemie, Weinheim, vol. 5.

In other embodiments, it is contemplated that lipases are used for the bioscouring of textiles either alone or with other bioscouring enzymes of the present compositions and methods. Suitable lipases (also, termed carboxylic ester hydrolases) include, without limitation, those of bacterial or fungal origin, including triacylglycerol lipases (3.1.1.3) and Phospholipase A2 (3.1.1.4.). Lipases include, without limitation, lipases from Humicola (synonym Thermomyces), such as from H. lanuginosa (T. lanuginosus) as described in patents or published patent applications EP 258,068 and EP 305,216 or from H. insolens as described in WO 96/13580; a Pseudomonas lipase, such as from P. alcaligenes or P. pseudoalcaligenes (EP 218,272), P. cepacia (EP 331,376), P. stutzeri (GB 1,372,034), P. fluorescens, Pseudomonas sp. strain SD 705 (WO 95/06720 and WO 96/27002), P. wisconsinensis (WO 96/12012); a Bacillus lipase, such as from B. subtilis (Dartois et al. (1993) Biochem. Biophys. Acta 1131:253-360); B. stearothermophilus (JP 64/744992) or B. pumilus (WO 91/16422), all references are herein incorporated by reference. Other examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079 and WO 97/07202, all of which are incorporated herein by reference. Preferred commercially available lipase enzymes include LIPOLASE™ and LIPOLASE ULTRA™, LIPOZYME™, PALATASE™, NOVOZYM™ 435 and LECITASE™ (all available from Novo Nordisk A/S). The activity of the lipase can be determined as described in “Methods of Enzymatic Analysis”, Third Edition, 1984, Verlag Chemie, Weinhein, vol. 4.

It will be understood that any enzyme exhibiting bioscouring activity may be used in practicing the present compositions and methods. That is, bioscouring enzymes derived from other organisms, or bioscouring enzymes derived from the enzymes listed above in which one or more amino acids have been added, deleted, or substituted, including hybrid polypeptides, may be used, so long as the resulting polypeptides exhibit bioscouring activity. Such variants can be created using conventional mutagenesis procedures and identified using, e.g., high-throughput screening techniques such as the agar plate screening procedure. For example, pectate lyase activity may be measured by applying a test solution to 4 mm holes punched out in agar plates (such as, for example, LB agar), containing 0.7% w/v sodium polygalacturonate (Sigma P 1879). The plates are then incubated for 6 h at a particular temperature (such as, e.g., 75° C.). The plates are then soaked in either (i) 1 M CaCl₂ for 0.5 h or (ii) 1% mixed alkyl trimethylammonium Br (MTAB, Sigma M-7635) for 1 h. Both of these procedures cause the precipitation of polygalacturonate within the agar. Pectate lyase activity can be detected by the appearance of clear zones within a background of precipitated polygalacturonate. Sensitivity of the assay is calibrated using dilutions of a standard preparation of pectate lyase.

### Desizing Enzymes

In some embodiments, the methods for enzymatic textile bleaching described herein include one or more desizing enzyme. One or more desizing enzyme may be included in the enzymatic textile bleaching composition, or a textile may be treated with desizing enzyme(s) in a subsequent processing step after pretreatment in the enzymatic textile bleaching composition.

Any suitable desizing enzyme may be used in the present compositions and methods. In some embodiments, the desizing enzyme is an amylolytic enzyme. Mannanases and glucoamylases may also be used. In some embodiments, the desizing enzyme is an α- or β-amylase and combinations thereof.

Amylases

Alpha and beta amylases, which are appropriate in the context of the present compositions and methods, include those of bacterial or fungal origin. Chemically or genetically modified mutants of such amylases are also included in this connection. Preferred α-amylases include, for example, α-amylases obtainable from Bacillus species. Useful amylases include but are not limited to OPTISIZE 40™, OPTISIZE 160™, OPTISIZE HT 260™, OPTISIZE HT 520™, OPTISIZE HT Plus™, OPTISIZE FLEX™ (all from Genencor), DURAMYL™, TERMAMYL™, FUNGAMYL™ and BAN™ (all available from Novozymes A/S, Bagsvaerd, Denmark). Other preferred amylolytic enzymes are CGTases (cyclodextrin glucanotransferases, EC 2.4.1.19), e.g., those obtained from species of Bacillus, Thermoanaerobactor or Thermoanaero-bacterium.

The activity of OPTISIZE 40™ and OPTISIZE 160™ is expressed in RAU/g of product. One RAU is the amount of enzyme which will convert 1 gram of starch into soluble sugars in one hour under standard conditions. The activity of OPTISIZE HT 260™, OPTISIZE HT 520™ and OPTISIZE HT Plus™ is expressed in TTAU/g. One TTAU is the amount of enzyme that is needed to hydrolyze 100 mg of starch into soluble sugars per hour under standard conditions. The activity of OPTISIZE FLEX™ is determined in TSAU/g. One TSAU is the amount of enzyme needed to convert 1 mg of starch into soluble sugars in one minute under standard conditions.

Dosage of the amylase varies depending on the process type. Smaller dosages would require more time than larger dosages of the same enzyme. However, there isn't an upper limit on the amount of desizing amylase other than what may be dictated by the physical characteristics of the solution. Excess enzyme does not hurt the fabric; it allows for a shorter processing time. Based on the foregoing and the enzyme utilized the following minimum dosages for desizing are suggested:

| | Minimum dosage | Typical Range |
|---|---|---|
| | (per liter of | (per liter of |
| Amylase Product | desizing liquor) | desizing liquor) |
| OPTISIZE 40 ™ | 1,000 RAU | 2,000-70,000 RAU |
| OPTISIZE 160 ™ | 1,000 RAU | 2,000-70,000 RAU |
| OPTISIZE HT 26 ™ 0 | 1,000 TTAU | 3,000-100,000 TTAU |
| OPTISIZE HT 520 ™ | 1,000 TTAU | 3,000-100,000 TTAU |
| OPTISIZE HT Plus ™ | 1,000 TTAU | 3,000-100,000 TTAU |
| OPTISIZE FLEX ™ | 5,000 TSAU | 13,000-65,000 TSAU |

The desizing enzymes may be derived from the enzymes listed above in which one or more amino acids have been added, deleted, or substituted, including hybrid polypeptides, so long as the resulting polypeptides exhibit desizing activity. Such variants useful in practicing the present compositions and methods can be created using conventional mutagenesis procedures and identified using, e.g., high-throughput screening techniques such as the agar plate screening procedure.

The desizing enzyme is added to the aqueous solution (i.e., the treating composition) in an amount effective to desize the textile materials. Typically, desizing enzymes, such as α-amylases, are incorporated into the treating composition in amount from about 0.00001% to about 2% of enzyme protein by weight of the fabric, preferably in an amount from about 0.0001% to about 1% of enzyme protein by weight of the fabric, more preferably in an amount from about 0.001% to about 0.5% of enzyme protein by weight of the fabric, and even more preferably in an amount from about 0.01% to about 0.2% of enzyme protein by weight of the fabric.

### Textiles

The present compositions and methods provide textiles, e.g., bleached textiles, produced according to any of the enzymatic bleaching methods described herein. Bleached textiles produced by incubation with enzymatic textile bleaching compositions as described herein exhibit at least one of decreased textile damage, increased dye uptake, and bulkier softer handle when compared to bleached textiles prepared with a chemical bleaching composition that does not contain the perhydrolase enzyme. The present compositions and methods also provides dyed textiles produced from bleached textiles that have been produced according to the enzymatic bleaching methods herein.

In some embodiments, the bleached and/or bleached and dyed textile is a cellulose-containing textile, including but not limited to cotton, flax, hemp, ramie, cellulose acetate, lyocell, viscose rayon, bamboo, and various cellulosic blends. In some embodiments, the bleached and/or bleached and dyed textile is a polyamide, polyacrylic, or wool textile, or a blend thereof.

### Kits

The compositions and methods may be provided in the form of a kit of parts (i.e., a kit). In one embodiment, the kit provides perhydrolase enzyme, with instructions for use of the perhydrolase enzyme in an enzymatic textile bleaching composition and/or enzymatic textile bleaching method as described herein. Suitable packaging is provided. As used herein, “packaging” refers to a solid matrix or material customarily used in a system and capable of holding within fixed limits components of a kit as described herein, e.g., perhydrolase enzyme.

Instructions may be provided in printed form or in the form of an electronic medium such as a floppy disc, CD, or DVD, or in the form of a website address where such instructions may be obtained.

The following examples are intended to illustrate, but not limit, the present compositions and methods.

### EXAMPLES

### Example 1

### Enzymatic Bleach Pretreatment of 100% Cotton Single Jersey Material

A comparison between enzymatic and chemical bleaching processes was performed using cotton jersey textile material in a batch process in a Mathis AG Lab Jet apparatus.

### Bleaching Compositions

The compositions shown in Table 1 were used in experiments as described below.

TABLE 1
Bleaching Compositions

| Bleaching | | | | | |
|---|---|---|---|---|---|
| Composition | | 1 | 2 | 3 | 4 |
| Clarite ® ONE | ml/l | 1.5 | 1.5 | 1.5 | 1.5 |
| Phosphate Buffer, | ml/l | | | 10 | 10 |
| pH 8 | | | | | |
| Propylene Glycol | ml/l | | | 3.0 | 3.0 |
| Diacetate | | | | | |
| Pectinase | ml/l | | | | 2.5 |
| NaOH 100% | g/l | 1.5 | 1.5 | | |
| H₂O₂ 35% | ml/l | 6.0 | 4.0 | 6.0 | 6.0 |
| Perhydrolase | g/l | | | 1.0 | 1.0 |

CLARITE ® ONE contained the following components: 0.5% (w/w) phosphonic acid [[(phosphonomethyl)imino]bis[2,1-ethanediyl nitrilobis(methylene)]]tetrakis-, sodium salt 5-10% (w/w) alkylethoxylate 15-20% (w/w) isotridecanol, ethoxylated <5% (w/w) polyacrylic acid, sodium salt
The phosphate buffer contained 10% soda ash.
The pectinase was a 10% solution of BIOPREP ™ 3000L, available from Novozymes.
The perhydrolase was the S54V variant of SEQ ID NO: 1 at a stock concentration of 1.7 g/l.

### Pretreatment Process

About 120 g of fabric was incubated in each pretreatment composition with a liquor ratio of about 10:1. The MathisAG Laboratory Jet machine raised the temperature of the bath by 3° C. per minute from ambient temperature to a target temperature of 65° C. The bath was then held at 65° C. for 50 minutes.

Two rinses were performed for 10 minutes each at 50° C. A 25% solution of CATALASE T100™, available from Genencor, was included in each rinse. Peroxide concentration before and after rinsing is shown in Table 2 for each bleaching composition tested. Peroxide concentration was assessed using indicator strips from Merck.

TABLE 2
Peroxide Concentration Before and After Rinsing with Catalase

| Bleaching | | | | | |
|---|---|---|---|---|---|
| Composition | | 1 | 2 | 3 | 4 |
| Before | ppm | 25 | 25 | 20 | 15 |
| After | ppm | 0 | 0 | 0 | 0 |

### Rewetting

Rewetting was assessed for fabric treated with each bleaching composition described above using a modified wicking test. Deionized water was placed in a beaker, a strip of fabric was added to the beaker just touching the water, and the time was then measured for the water to travel 1 cm. Better hydrophilicity is indicated by a low rewetting rate, expressed in cm/sec. The results are shown in Table 3.

TABLE 3
Rewetting Values

| Bleaching | | | | |
|---|---|---|---|---|
| Composition | 1 | 2 | 3 | 4 |
| Hydrophilicity | 1.5 | 2.7 | 104 | 80 |
| (cm/sec) | | | | |

### Whiteness

Whiteness was quantified using four different test methods. The results are shown in Table 4.

TABLE 4
Degree of Whiteness

| Bleaching | | | | |
|---|---|---|---|---|
| Composition | 1 | 2 | 3 | 4 |
| Ganz | 50 | 46 | 25 | 24 |
| ISO/Tappi | 86.0 | 85.4 | 80.1 | 78.9 |
| CIE | 73 | 71 | 60 | 58 |
| Berger | 72 | 69 | 59 | 58 |

### Fabric Damage Assessment

Degree of polymerization was assessed for fabric treated with each bleaching composition described above. Degree of polymerization was determined using the Swiss EWN Method (Swiss standard SNV 195 598). The damage factor (S) was determined according to the formula from O. Eisenhut, relating fiber damage to the change in degree of polymerization value before and after pretreatment.

The results are presented in Table 5. For comparison, the degree of polymerization for grey 100% cotton knitgood was 2380.

TABLE 5
Fabric Damage Assessment

| Bleaching | | | | |
|---|---|---|---|---|
| Composition | 1 | 2 | 3 | 4 |
| Degree of | 2060 | 2110 | 2280 | 2230 |
| Polymerization | | | | |
| Damage Factor | s: 0.17 | s: 0.15 | s: 0.05 | s: 0.08 |

### Dyeing and Color Fastness

Fabric treated with the bleaching compositions described above was dyed with NOVACRON® Rot FN 3G, 3% (w/w), for 90 min at 60° C. in a MathisAG Labomat device. Dye depth, hue deviation, and chroma deviation were assessed.

The results of the colorimetric assessment are shown in Table 6. Colorimetric assessment was based on the colorimetry CIE-Lab (Munsell0 with hue deviation indicating differences in shade (red-green and blue-yellow) and chroma deviation indicating differences in brilliancy.

TABLE 6
Colorimetric Assessment

| Bleaching | | | | |
|---|---|---|---|---|
| Composition | 1 | 2 | 3 | 4 |
| Relative Dye | 100 | 100 | 100 | 104 |
| Depth | | | | |
| Hue Deviation | — | −0.2 | −0.4 | −0.3 |
| | | | (trace redder) | (trace redder) |
| Chroma | — | −0.3 | −0.1 | −0.6 |
| Deviation | | (trace duller) | | (slightly duller) |

Fastness was assessed as rubbing fastness, wash fastness, water fastness, and perspiration acid and alkaline fastness. Wet/dry rubbing fastness (crocking) was assessed according to test method ISO 105-X12. Wash fastness was assessed at 60° C. according to test method ISO 105-C06. Water fastness was assessed according to test method ISO 105-E01. Acid/alkaline perspiration fastness was assessed according to test method ISO 105-E04. For all of these parameters, similar results were obtained for the chemical bleaching compositions (1 and 2) and the enzymatic bleaching compositions (3 and 4).

### Handle

A bulkier, softer fabric handle was observed with fabric that was pretreated in the enzymatic bleaching compositions (3 and 4) in comparison to fabric pretreated in the chemical bleaching compositions (1 and 2), both before and after dyeing.

Although the foregoing present compositions and methods have been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention.

All publications, patents, and patent applications cited herein are hereby incorporated by reference in their entireties for all purposes and to the same extent as if each individual publication, patent, or patent application were specifically and individually indicated to be so incorporated by reference.

1. An enzymatic textile bleaching composition, comprising:
(i) a perhydrolase enzyme;
(ii) an ester substrate for said perhydrolase enzyme;
(iii) a hydrogen peroxide source;
(iv) a surfactant and/or an emulsifier;
(v) a peroxide stabilizer;
(vi) a sequestering agent; and
(vii) a buffer that maintains a pH of about 6 to about 8.

2. The enzymatic textile bleaching composition of claim 1, wherein the perhydrolase enzyme comprises the amino acid sequence set forth in SEQ ID NO: 1 or a variant or homolog thereof.

3. The enzymatic textile bleaching composition of claim 2, wherein the perhydrolase enzyme is the S54V variant of SEQ ID NO: 1.

4. The enzymatic textile bleaching composition of claim 1, wherein the perhydrolase enzyme exhibits a perhydrolysis to hydrolysis ratio greater than 1.

5. The enzymatic textile bleaching composition of claim 1, wherein the perhydrolase enzyme is present at a concentration of about 1 to about 2.5 ppm.

6. The enzymatic textile bleaching composition of claim 1, wherein the ester substrate is selected from propylene glycol diacetate, ethylene glycol diacetate, triacetin, ethyl acetate, and tributyrin.

7. The enzymatic textile bleaching composition of claim 6, wherein the ester substrate is propylene glycol diacetate.

8. The enzymatic textile bleaching composition of claim 7, wherein propylene glycol diacetate is present is present in the composition in an amount of about 2,000 to about 4,000 ppm.

9. The enzymatic textile bleaching composition of claim 1, wherein the hydrogen peroxide source is hydrogen peroxide.

10. The enzymatic textile bleaching composition of claim 9, wherein hydrogen peroxide is present at a concentration of about 1,000 to about 3,000 ppm.

11. The enzymatic textile bleaching composition of claim 1, wherein the surfactant and/or emulsifier comprises a non-ionic surfactant.

12. The enzymatic textile bleaching composition of claim 12, wherein the non-ionic surfactant is an alcohol ethoxylate.

13. The enzymatic textile bleaching composition of claim 1, wherein the surfactant and/or emulsifier comprises an isotridecanol ethoxylate.

14. The enzymatic textile bleaching composition of claim 1, wherein the surfactant and/or emulsifier comprises an alcohol ethoxylate and an isotridecanol ethoxylate.

15. The enzymatic textile bleaching composition of claim 1, comprising a surfactant and an emulsifier.

16. The enzymatic textile bleaching composition of claim 1, wherein the peroxide stabilizer is phosphonic acid.

17. The enzymatic textile bleaching composition of claim 1, wherein the sequestering agent is polyacrylic acid.

18. The enzymatic textile bleaching composition of claim 1, further comprising a bioscouring enzyme.

19. The enzymatic textile bleaching composition of claim 18, wherein the bioscouring enzyme is selected from pectinases, cutinases, cellulases, hemicellulases, proteases, and lipases.

20. The enzymatic textile bleaching composition of claim 18, wherein the bioscouring enzyme is a pectinase.

21. A method for bleaching a textile, comprising contacting the textile with an enzymatic textile bleaching composition according to claim 1 for a length of time and under conditions suitable to permit measurable whitening of the textile, thereby producing a bleached textile, wherein the bleached textile comprises at least one of decreased textile damage, bulkier softer handle, and increased dye uptake when compared to a chemical textile bleaching method that comprises contacting the textile with a chemical textile bleaching composition that does not comprise a perhydrolase enzyme.

22. The method of claim 21, further comprising hydrolyzing hydrogen peroxide with a catalase enzyme after the bleached textile is produced.

23. The method of claim 21, wherein the liquor ratio is about 10:1.

24. The method of claim 21 performed in a batch or exhaust process.

25. The method of claim 21, wherein the method provides any of at least about 10, 20, 30, 40, or 50% less weight loss than a chemical bleaching composition that does not comprise a perhydrolase enzyme.

26. The method of claim 21, wherein the method provides a textile capable of increased dye uptake to produce a dyed textile with at least about any of at least about 5, 10, 15, 20, 25, or 30% increased dye depth when compared to a textile treated with a chemical bleaching composition that does not comprise a perhydrolase enzyme.

27. The method of claim 21, wherein the method provides a textile that exhibits reduced pilling propensity when compared to a textile treated with a chemical bleaching composition that does not comprise a perhydrolase enzyme.

28. The method of claim 21, wherein the textile is contacted with the enzymatic textile bleaching composition at a bleaching temperature of about 60° to about 70° C. for a processing time of about 40 to about 60 minutes.

29. The method of claim 28, wherein the temperature of the enzymatic textile bleaching composition is raised by about 3° C. per minute from a starting temperature of about 20° to about 40° C. until the bleaching temperature is reached.

30. The method of claim 28, wherein the bleaching temperature is about 65° C. and the processing time is about 50 minutes.

31. The method of claim 21, wherein the bleached textile is rinsed with an aqueous composition at a rinsing temperature of about 40° C. to about 60° C. to remove said enzymatic textile bleaching composition.

32. The method of claim 31, wherein the rinsing temperature is about 50° C.

33. The method of claim 31, wherein the rinsing comprises rinsing said bleached textile twice for about 10 minutes for each rinse.

34. The method of claim 21, wherein the aqueous composition comprises a catalase enzyme to hydrolyze the hydrogen peroxide.

35. (canceled)
